(19) United States
(12) Patent Application Publication
(10) Pub. No.: US 2026/0076680 A1
(43) Pub. Date: Mar. 19, 2026

(54) LEFT ATRIAL APPENDAGE CLOSURE DEVICE WITH OCCLUSIVE MEMBER RETENTION FEATURE

(71) Applicant: BOSTON SCIENTIFIC SCIMED, INC., Maple Grove, MN (US)

(72) Inventors: Neal Eidenschink, Wayzata, MN (US); Daniel Ross, Watertown, MN (US); Christopher J Clark, St. Michael, MN (US); Thyna Michael Chau, Rochester, MN (US); Harishankar Natesan, Minneapolis, MN (US)

(73) Assignee: BOSTON SCIENTIFIC SCIMED, INC., MAPLE GROVE, MN (US)

(21) Appl. No.: 19/328,999

(22) Filed: Sep. 15, 2025

Related U.S. Application Data
(60) Provisional application No. 63/695,101, filed on Sep. 16, 2024.

(51) Int. Cl. A61B 17/12; A61B 17/00

(52) U.S. Cl. CPC A61B 17/12122; A61B 17/12172; A61B 2017/00477

(57) ABSTRACT

A left atrial appendage closure (LAAC) device includes an expandable frame that is adapted to shift between a collapsed configuration and an expanded configuration, the expandable frame comprising a plurality of expandable frame struts and an occlusive member that is disposed over the expandable frame. The LAAC device includes a plurality of anchor members that are each coupled with a corresponding one of the plurality of expandable frame struts. Each of the plurality of anchor members includes an occlusive member retention region having a first occlusive member retention feature and an associated second occlusive member retention feature.

### CROSS REFERENCE TO RELATED APPLICATIONS

This application claims the benefit of priority of U.S. Provisional Application No. 63/695,101 filed Sep. 16, 2024, the entire disclosure of which is hereby incorporated by reference.

### TECHNICAL FIELD

The disclosure relates generally to medical devices and more particularly to left atrial appendage closure devices.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device. The LAAC device includes an expandable frame that is adapted to shift between a collapsed configuration and an expanded configuration and an occlusive member that is disposed over the expandable frame. The expandable frame includes a plurality of expandable frame struts. The LAAC device includes a plurality of anchor members that are each coupled with a corresponding one of the plurality of expandable frame struts. Each of the plurality of anchor members include an anchor member base that extends laterally from the corresponding expandable frame strut, an anchor member strut that extends axially from the anchor member base and defines a space between the anchor member strut and the corresponding expandable frame strut, and one or more pairs of opposing barbs that extend into the space, each pair of opposing barbs including one barb extending into the space from the anchor member strut and one barb extending into the space from the corresponding expandable frame strut.

Alternatively or additionally, for at least some of the plurality of anchor members, the anchor member strut may extend at least substantially parallel with the expandable frame strut.

Alternatively or additionally, for at least some of the plurality of anchor members, the anchor member struts may be at least substantially coplanar with the expandable frame strut.

Alternatively or additionally, at least some of the plurality of anchor members may further include a tip portion that extends from an end of the anchor member strut opposite to the anchor member base.

Alternatively or additionally, for at least some of the plurality of anchor members, the tip portion may curve away from the anchor member strut.

Alternatively or additionally, for at least some of the plurality of anchor members, the tip portion may be adapted to engage tissue when the LAAC device is implanted.

Alternatively or additionally, the one or more pairs of opposing barbs may include two pairs of opposing barbs.

Alternatively or additionally, the one or more pairs of opposing barbs may include three or more pairs of opposing barbs.

Alternatively or additionally, the one or more pairs of opposing barbs may be adapted to engage with the occlusive member.

Another example may be found in a left atrial appendage closure (LAAC) device. The LAAC device includes an expandable frame that is adapted to shift between a collapsed configuration and an expanded configuration. The expandable frame includes a plurality of expandable frame struts. An occlusive member is disposed over the expandable frame. The LAAC device includes a plurality of anchor members that are each coupled with a corresponding one of the plurality of expandable frame struts. Each of the plurality of anchor members include an anchor member base that extends laterally from the corresponding expandable frame strut, an anchor member strut that extends axially from the anchor member base and is substantially parallel with the corresponding expandable frame strut, a tissue engaging region that extends from the anchor member strut, and one or more pairs of opposing barbs. Each pair of opposing barbs includes a first barb extending towards the corresponding expandable frame strut from the anchor member strut and a second barb extending towards the anchor member strut from the corresponding expandable frame strut.

Alternatively or additionally, the anchor member strut may be spaced apart from the corresponding expandable frame strut such that the anchor member strut and the corresponding expandable frame strut define a space therebetween.

Alternatively or additionally, the first barb and the second barb may be at least substantially coplanar.

Another example may be found in a left atrial appendage closure (LAAC) device. The LAAC device includes an expandable frame that includes a plurality of expandable frame struts and is adapted to shift between a collapsed configuration and an expanded configuration. An occlusive member is disposed over the expandable frame. The LAAC device includes a plurality of anchor members that are each coupled with a corresponding one of the plurality of expandable frame struts. Each of the plurality of anchor members include an occlusive member retention region including a first occlusive member retention feature and an associated second occlusive member retention feature.

Alternatively or additionally, each of the plurality of anchor members may include an anchor member strut spaced apart from the corresponding expandable frame strut.

Alternatively or additionally, the first occlusive member retention feature may extend at a first angle relative to one of the anchor member strut and the corresponding expandable frame strut and the second occlusive member retention feature may extend from the first occlusive member retention feature at a second angle different from the first angle.

Alternatively or additionally, the first occlusive member retention feature may include a first barb that extends from the anchor member strut towards the corresponding expandable frame strut and the second occlusive member retention feature may include a second barb extending from the corresponding expandable frame strut towards the anchor member strut.

Alternatively or additionally, the first barb may be axially spaced from the second barb.

Alternatively or additionally, the first occlusive member retention feature may include a barb that extends from one of the anchor member strut and the corresponding frame strut, and the second occlusive member retention may include a complementary recess that is formed in the other of the anchor member strut and the corresponding frame strut.

Alternatively or additionally, the first occlusive member retention feature may partially extend into the second occlusive member feature.

Alternatively or additionally, at least some of the anchor members may further include a tissue engaging region that curls relative to the corresponding expandable strut.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an illustrative LAAC device;

FIG. 2 is a side view of an illustrative expandable frame forming part of the illustrative LAAC device of FIG. 1;

FIG. 3 is an enlarged view of a portion of an illustrative distal anchor member forming part of the illustrative expandable frame of FIG. 2;

FIG. 4 is an enlarged view of a portion of an illustrative distal anchor member of FIG. 3 including an occlusive member retention region;

FIG. 5 is an enlarged view of a portion of an illustrative distal anchor member of FIG. 3 including an occlusive member retention region;

FIG. 6 is an enlarged view of a portion of an illustrative distal anchor member of FIG. 3 including an occlusive member retention region;

FIG. 7 is an enlarged view of a portion of an illustrative distal anchor member of FIG. 3 including an occlusive member retention region;

FIG. 8 is an enlarged view of a portion of an illustrative distal anchor member of FIG. 3 including an occlusive member retention region; and

FIG. 9 is an enlarged view of a portion of an illustrative distal anchor member of FIG. 3 including an occlusive member retention region.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A left atrial appendage closure (LAAC) device may be used to close off or otherwise occlude the volume within a left atrial appendage from the rest of the left atrium. Preventing blood flow in and out of the left atrial appendage can reduce the possibility of embolisms from forming within the left atrial appendage and reaching other parts of the body, including the brain. In some instances, a left atrial appendage closure (LAAC) device includes an expandable frame that is adapted to shift between a collapsed configuration and an expanded configuration. The expandable frame includes a plurality of expandable frame struts. An occlusive member is disposed over the expandable frame. The LAAC device includes a plurality of anchor members that are each coupled with a corresponding one of the plurality of expandable frame struts. Each of the plurality of anchor members include an anchor member base extending laterally from the corresponding expandable frame strut, an anchor member strut extending axially from the anchor member base and defining a space between the anchor member strut and the corresponding expandable frame strut, and one or more pairs of opposing barbs extending into the space, each pair of opposing barbs including one barb extending into the space from the anchor member strut and one barb extending into the space from the corresponding expandable frame strut.

In some cases, for at least some of the plurality of anchor members, the anchor member strut may extend at least substantially parallel with the expandable frame strut, where substantially parallel is defined as the anchor member strut and the expandable frame strut being within twenty percent or less of parallel, or within ten percent or less of parallel. In some cases, for at least some of the plurality of anchor members, the anchor member struts may be at least substantially coplanar with the expandable frame strut, where substantially coplanar is defined as the anchor member strut and the expandable frame strut may be within twenty percent or less of coplanar, or within ten percent or less of coplanar.

In some cases, at least some of the plurality of anchor members may further include a tip portion that extends from an end of the anchor member strut opposite to the anchor member base. For at least some of the plurality of anchor members, the tip portion may curve away from the anchor member strut. For at least some of the plurality of anchor members, the tip portion may be adapted to engage tissue when the LAAC device is implanted. In some cases, the one or more pairs of opposing barbs may include two pairs of opposing barbs, or three pairs of opposing barbs. In some cases, the one or more pairs of opposing barbs may be adapted to engage with the occlusive member.

In some instances, a left atrial appendage closure (LAAC) device includes an expandable frame that includes a plurality of expandable frame struts and that is adapted to shift between a collapsed configuration and an expanded configuration. An occlusive member is disposed over the expandable frame. A plurality of anchor members are each coupled with a corresponding one of the plurality of expandable frame struts. Each of the plurality of anchor members include an anchor member base extending laterally from the corresponding expandable frame strut, an anchor member strut extending axially from the anchor member base and substantially parallel with the corresponding expandable frame strut, a tissue engaging region extending from the anchor member strut, and one or more pairs of opposing barbs, each pair of opposing barbs including a first barb extending towards the corresponding expandable frame strut towards the anchor member strut and a second barb extending towards the anchor member strut from the corresponding expandable frame strut.

In some cases, the anchor member strut may be spaced apart from the corresponding expandable frame strut such that the anchor member strut and the corresponding expandable frame strut define a space therebetween. In some cases, the first barb and the second barb may be at least substantially coplanar. Substantially coplanar may be defined as the first barb and the second barb being within twenty percent or less of coplanar, or within ten percent or less of coplanar.

In some instances, a left atrial appendage closure (LAAC) device includes an expandable frame that is adapted to shift between a collapsed configuration and an expanded configuration, the expandable frame including a plurality of expandable frame struts. An occlusive member is disposed over the expandable frame, and a plurality of anchor members are each coupled with a corresponding one of the plurality of expandable frame struts, each of the plurality of anchor members including an occlusive member retention region including a first occlusive member retention feature and an associated second occlusive member retention feature.

In some cases, each of the plurality of anchor members may include an anchor member strut that is spaced apart from the corresponding expandable frame strut. In some cases, the first occlusive member retention feature may extend at a first angle relative to one of the anchor member strut and the corresponding expandable frame strut, and the second occlusive member retention feature may extend from the first occlusive member retention feature at a second angle different from the first angle. In some cases, the first occlusive member retention feature may include a first barb that extends from the anchor member strut towards the corresponding expandable frame strut and the second occlusive member retention feature may include a second barb that extends from the corresponding expandable frame strut towards the anchor member strut. The first barb may be axially spaced from the second barb, for example.

In some cases, the first occlusive member retention feature may include a barb that extends from one of the anchor member strut and the corresponding frame strut, and the second occlusive member retention may include a complementary recess that is formed in the other of the anchor member strut and the corresponding frame strut. In some cases, the first occlusive member retention feature may partially extend into the second occlusive member feature. In some cases, at least some of the anchor members may further include a tissue engaging region that curls relative to the corresponding expandable strut.

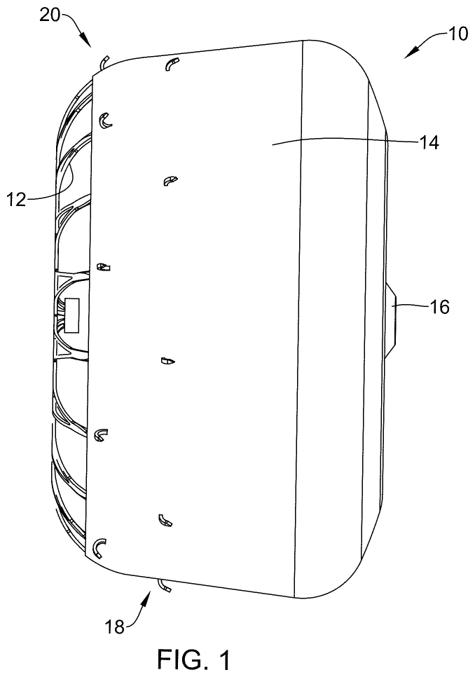

FIG. 1 is a side view of an illustrative LAAC device 10 that may be adapted to be implanted within a patient's LAA in order to occlude the patient's LAA and thus reduce or even eliminate the possibility for a blood clot within the LAA to exit the LAA and enter the patient's vasculature. An exemplary LAAC device 10 includes WATCHMAN FLX™ and WATCHMAN FLX™ Pro available from Boston Scientific.

The LAAC device 10 may include an expandable frame 12 and an occlusive member 14 that extends over at least part of the expandable frame 12. The expandable frame 12 (and hence the occlusive member 14) may be movable between a collapsed configuration for delivery and an expanded configuration (as shown) when deployed. The LAAC device 10 may be adapted to be implanted within a patient's left atrial appendage in order to reduce or even eliminate blood flow into or out of the left atrial appendage. The expandable frame 12 may define a hub 16 that allows for a delivery device (not shown) to be releasably secured to the LAAC device 10 for delivery and deployment. The hub 16 is adapted to allow a delivery device to be disconnected after the LAAC device 10 has been deployed, allowing the LAAC device 10 to remain behind in position within the patient's left atrial appendage.

In some examples, the expandable frame 12 may be integrally formed and/or cut from a unitary member. In some cases, the expandable frame 12 and may be integrally formed and/or cut from a unitary tubular member and subsequently formed and/or heat set to a desired shape in the expanded configuration. In some cases, the expandable frame 12 may be integrally formed and/or cut from a unitary flat member, and then rolled or formed into a tubular structure and subsequently formed and/or heat set to the desired shape in the expanded configuration. Some exemplary means and/or methods of making and/or forming the expandable frame 12 include laser cutting, machining, punching, stamping, electro discharge machining (EDM), chemical dissolution, etc. Other means and/or methods are also contemplated.

In some cases, the occlusive member 14 may be permeable or impermeable to blood and/or other fluids, such as water. In some cases, the occlusive member 14 may prevent thrombi (i.e. blood clots, etc.) from passing through the occlusive member 14 and out of the LAA into the blood stream. In some cases, the occlusive member 14 may promote endothelialization after implantation, thereby effectively removing the left atrial appendage from the patient's circulatory system.

The occlusive member 14 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction. The occlusive member 14 may be formed from a suitable material such as polyethylene terephthalate, polyester, nylon, acrylic materials, a polyolefin, and/or the like, combinations thereof, and/or other materials disclosed herein. In other instances, the occlusive material may include metallic mesh formed from nickel-titanium alloy, stainless steel, titanium, other materials disclosed herein, combinations thereof, and/or the like.

In some cases, the expandable frame 12 may include tissue engaging features that extend through the occlusive member 14 in order to be able to engage tissue once the LAAC device 10 has been implanted and expanded. In some cases, as shown, the expandable frame may include a first row of tissue engaging features 18 and a second row of tissue engaging features 20. In some cases, the first row of tissue engaging features 18 may be considered as being proximal of the second row of tissue engaging features 20. The second row of tissue engaging features 20 may be considered as being distal of the first row of tissue engaging features 18. In some cases, the tissue engaging features 18 and/or the tissue engaging features 20 may be considered as being part of anchor members that are formed as part of the expandable frame 12.

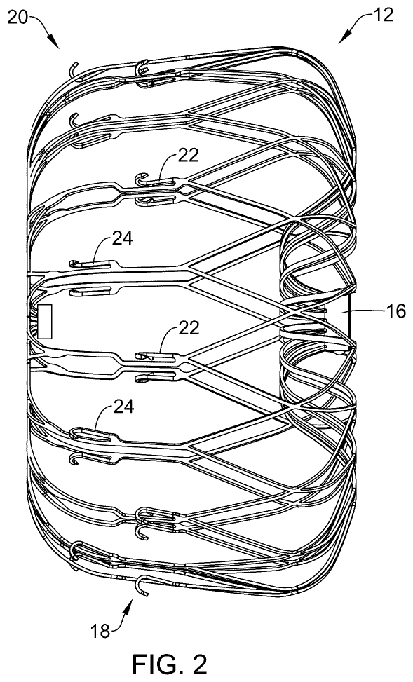

As seen in FIG. 2, which is a side view of the expandable frame 12 with the occlusive member 14 removed for clarity, it can be seen that the tissue engaging features 18 are part of anchor members 22 and the tissue engaging features 20 are part of anchor members 24. As shown, the anchor members 22 may be considered as being proximal anchor members and the anchor members 24 may be considered as being distal anchor members. The proximal anchor members 22 are shown arranged in a row extending circumferentially about the expandable frame 12. The distal anchor members 24 are shown arranged in a row extending circumferentially about the expandable frame 12. In some cases, at least some of the proximal anchor members 22 and/or at least some of the distal anchor members 24 may be displaced in either a proximal direction or a distal direction with respect to others of the proximal anchor members 22 and/or the distal anchor members 24.

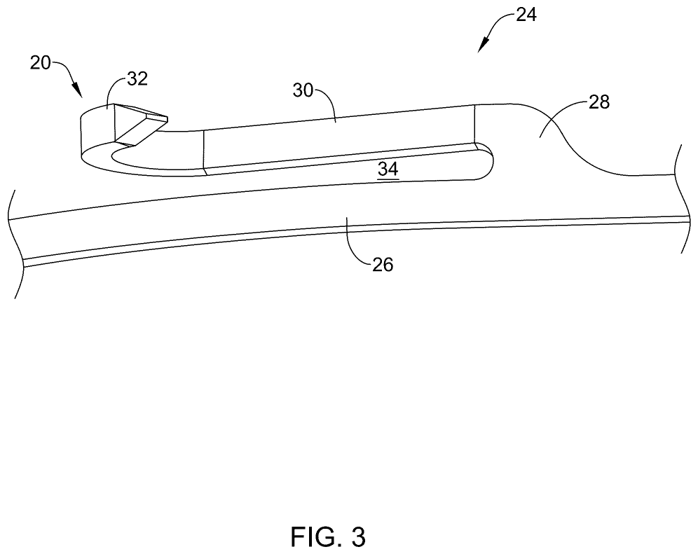

The distal anchor members 24 may be considered as not only including the aforementioned tissue engaging features 20, but also including features that are intended for securing the occlusive member 14 in position relative to the expandable frame 12. FIG. 3 is an enlarged view of one of the distal anchor members 24. The distal anchor member 24 includes an expandable frame strut 26 that is part of the expandable frame 12. It will be appreciated that the expandable frame 12 includes a plurality of expandable frame struts 26. The distal anchor member 24 includes an anchor base member 28 that extends laterally from the expandable frame strut 26. An anchor member strut 30 extends axially from the anchor member base 28. In some cases, the anchor member strut 30 may be considered as being at least substantially parallel with the expandable frame strut 26. In some cases, the anchor member strut 30 may be considered as being at least substantially coplanar with the expandable frame strut 26. In some cases, while not shown, the anchor member strut 30 may extend at an angle with respect to the expandable frame strut 26. As an example, the anchor member strut 30 may remain coplanar with the expandable frame strut 26 but may not be parallel to the expandable frame strut 26. In some cases, the anchor member strut 30 may be neither parallel nor coplanar with the expandable frame strut 26. For example, the anchor member strut 30 may either extend into the plane of the paper or may extend out of the plane of the paper.

In some cases, the distal anchor member 24 may include a tip portion 32 that is at an opposite end of the anchor member strut 30 from where the anchor member strut 30 is coupled with the anchor base member 28 and that curves away from the anchor member strut 30. The tip portion 32 may form the tissue engaging feature 20, for example. In some cases, the anchor base member 28 and the anchor member strut 30 are integrally formed as part of the expandable frame 12. As an example, the anchor base member 28 and the anchor member strut 30 may each be formed via laser cutting as part of the expandable frame 12.

A space 34 is defined between the expandable frame strut 26 and the anchor member strut 30. As shown in FIG. 3, the anchor member strut 30 is parallel and coplanar with the expandable frame strut 26, and thus the space 34 has a constant width. It will be appreciated that the space 34 may not have a constant width when the anchor member strut 30 is non-parallel with the expandable frame strut 26, and the space 34 may not have a constant width when the anchor member strut 30 is not coplanar with the expandable frame strut 26. In some cases, the space 34 may be adapted to engage the occlusive member 14 in order to secure the occlusive member 14 in place relative to the expandable frame 12. In some cases, the distal anchor member 24 may include occlusive member retention features that further help secure the occlusive member 14 in position relative to the expandable frame 12.

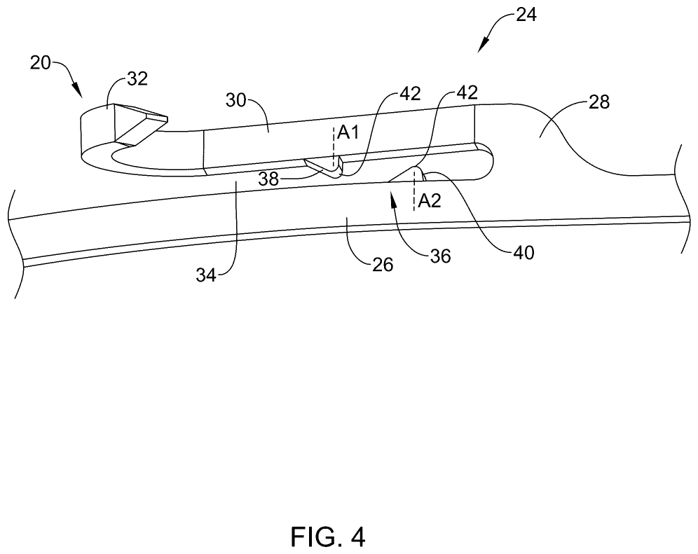

FIG. 4 shows the distal anchor member 24 as including an occlusive member retention region 36 that is adapted to help hold the occlusive member 14 relative to the expandable frame 12. The occlusive member retention region 36 may be laser cut as an integral part of the distal anchor member 24. The occlusive member retention region 36 includes a first occlusive member retention feature 38 that extends into the space 34 (and towards the expandable frame strut 26) from the anchor member strut 30 and a second occlusive member retention feature 40 that extends into the space 34 (and towards the anchor member strut 30) from the expandable frame strut 26. The first occlusive member retention feature 38 may be integrally formed with the anchor member strut 30 and the second occlusive member retention feature 40 may be integrally formed with the expandable strut frame 26. While the occlusive member retention region 36 is shown as only including the first occlusive member retention feature 38 and the second occlusive member retention feature 40, the occlusive member retention region 36 may include additional occlusive member retention features (not shown).

In some cases, the first occlusive member retention feature 38 and the second occlusive member retention feature 40 may be coplanar, such as within the plane of the paper. In some cases, the first occlusive member retention feature 38 may extend in a first direction relative to the plane of the paper and the second occlusive member retention feature 40 may extend in a second direction relative to the plane of the paper. As a example, one of the first occlusive member retention feature 38 and the second occlusive member retention feature 40 may extend upward out of the plane of the paper while the other of the first occlusive member retention feature 38 and the second occlusive member retention feature 40 may extend downward into the plane of the paper. The opposite may also be true. In some cases, both of the first occlusive member retention feature 38 and the second occlusive member retention feature 40 may both extend in the same direction, either into the plane of the paper or out of the plane of the paper.

In some cases, the first occlusive member retention feature 38 may be considered as including an axis A₁ extending orthogonally from the anchor member strut 30 and the second occlusive member retention feature 40 may be considered as including an axis A₂ extending orthogonally from the expandable frame strut 26. As shown, the first occlusive member retention feature 38 and the second occlusive member retention feature 40 may be considered as having a backward profile in which more of the first occlusive member retention feature 38 and more of the second occlusive member retention feature 40 is disposed to the left of the axis A₁ and A₂, respectively. A peak 42 of each of the first occlusive member retention feature 38 and the second occlusive member retention feature 40 may be closer to the anchor member base 28. In some cases, one or both of the first occlusive member retention feature 38 and the second occlusive member retention feature 40 may have a forward profile in which more of the first occlusive member retention feature 38 and more of the second occlusive member retention feature 40 is disposed to the right of the axis A₁ and A₂, respectively.

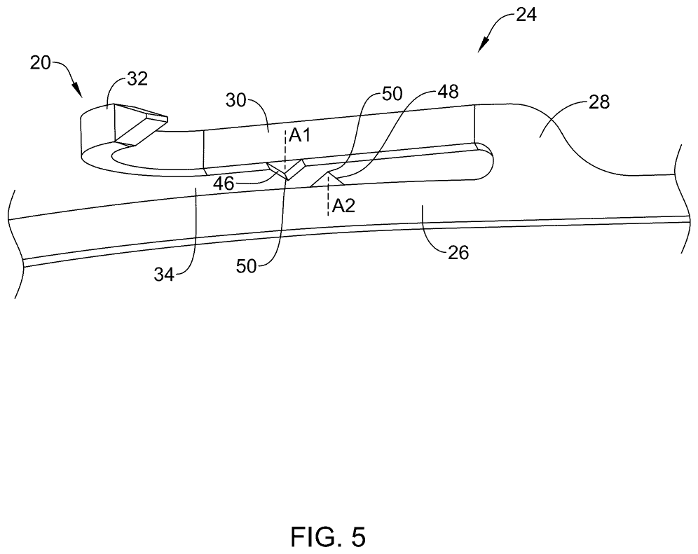

FIG. 5 shows the distal anchor member 24 as including an occlusive member retention region 44. The occlusive member retention region 44 may be laser cut as an integral part of the distal anchor member 24. The occlusive member retention region 44 includes a first occlusive member retention feature 46 that extends into the space 34 (and towards the expandable frame strut 26) from the anchor member strut 30 and a second occlusive member retention feature 48 that extends into the space 34 (and towards the anchor member strut 30) from the expandable frame strut 26. The first occlusive member retention feature 46 may be integrally formed with the anchor member strut 30 and the second occlusive member retention feature 48 may be integrally formed with the expandable strut frame 26. While the occlusive member retention region 44 is shown as only including the first occlusive member retention feature 46 and the second occlusive member retention feature 48, the occlusive member retention region 44 may include additional occlusive member retention features (not shown).

In some cases, the first occlusive member retention feature 46 and the second occlusive member retention feature 48 may be coplanar, such as within the plane of the paper. In some cases, the first occlusive member retention feature 46 may extend in a first direction relative to the plane of the paper and the second occlusive member retention feature 48 may extend in a second direction relative to the plane of the paper. As a example, one of the first occlusive member retention feature 46 and the second occlusive member retention feature 48 may extend upward out of the plane of the paper while the other of the first occlusive member retention feature 46 and the second occlusive member retention feature 48 may extend downward into the plane of the paper. The opposite may also be true. In some cases, both of the first occlusive member retention feature 46 and the second occlusive member retention feature 48 may both extend in the same direction, either into the plane of the paper or out of the plane of the paper.

In some cases, the first occlusive member retention feature 46 may be considered as including an axis A₁ extending orthogonally from the anchor member strut 30 and the second occlusive member retention feature 48 may be considered as including an axis A₂ extending orthogonally from the expandable frame strut 26. As shown, the first occlusive member retention feature 46 and the second occlusive member retention feature 48 may be considered as having a neutral profile in which the first occlusive member retention feature 46 and the second occlusive member retention feature 48 are centered on the axis A₁ and A₂, respectively. A peak 50 of each of the first occlusive member retention feature 46 and the second occlusive member retention feature 48 is centered on the first occlusive member retention feature 46 and the second occlusive member retention feature 48, respectively.

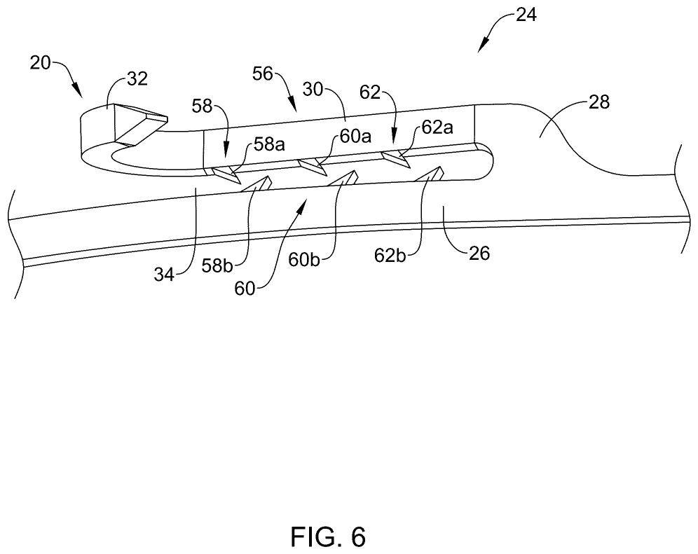

FIG. 6 shows the distal anchor member 24 as including an occlusive member retention region 56 that is adapted to help hold the occlusive member 14 relative to the expandable frame 12. The occlusive member retention region 56 may be laser cut as an integral part of the distal anchor member 24. The occlusive member retention region 56 includes a first pair of opposed barbs 58, a second pair of opposed barbs 60 and a third pair of opposed barbs 62. The first pair of opposed barbs 58 includes a first barb 58a extending towards the expandable frame strut 26 and a second barb 58b extending towards the anchor member strut 30. The second pair of opposed barbs 60 includes a first barb 60a extending towards the expandable frame strut 26 and a second barb 60b extending towards the anchor member strut 30. The third pair of opposed barbs 62 includes a first barb 62a extending towards the expandable frame strut 26 and a second barb 62b extending towards the anchor member strut 30. Each of the barbs 58a, 58b, 60a, 60b, 62a and 62b may be seen as having a backward profile. The distal anchor member 24 may include one barb, two barbs, three barbs, four barbs, or more in the occlusive member retention region 56. In some cases, the axial spacing between neighboring barbs or neighboring pairs of opposed barbs may vary. In some cases, the axial spacing between neighboring barbs or neighboring pairs of opposed barbs may be minimized in order to maximize occlusive member retention capability. In some cases, the axial spacing between neighboring barbs or neighboring pairs of opposed barbs may be a function of how many barbs or pairs of opposed barbs are present.

In some cases, each of the first pair of opposed barbs 58, the second pair of opposed barbs 60 and the third pair of opposed barbs 62 may independently be coplanar or at least substantially coplanar with the expandable frame strut 26 and the anchor member strut 30. In some cases, one or more of the first pair of opposed barbs 58, the second pair of opposed barbs 60 and the third pair of opposed barbs 62, or individual barbs 58a, 58b, 60a, 60b, 62a and 62b thereof, may independently extend in a direction into the plane of the paper or in a direction out of the plane of the paper.

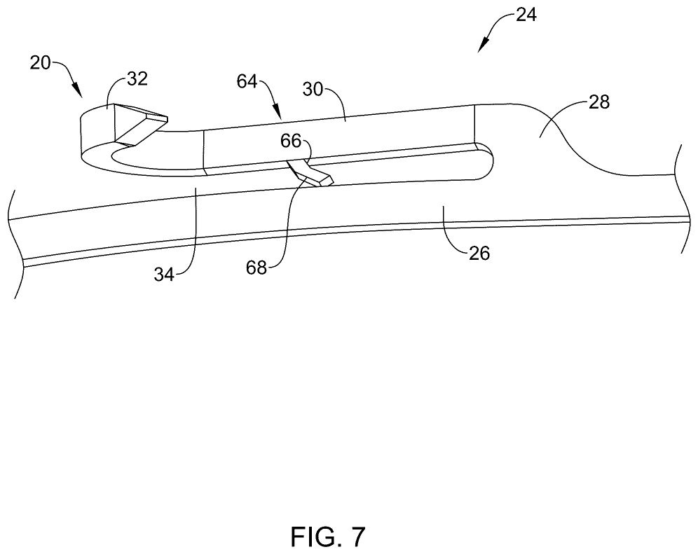

FIG. 7 shows the distal anchor member 24 as including an occlusive member retention region 64 that is adapted to help hold the occlusive member 14 relative to the expandable frame 12. The occlusive member retention region 64 may be laser cut as an integral part of the distal anchor member 24. As shown, the occlusive member retention region 64 includes a first occlusive member retention feature 66 that extends at a first angle relative to the anchor member strut 30 and a second occlusive member retention feature 68 that extends at a second angle different from the first angle. As a result, the first occlusive member retention feature 66 extends in a direction that is relatively closer to orthogonal to the anchor member strut 30 and the second occlusive member retention feature 68 extends in a direction that is closer to parallel to the anchor member strut 30. While the first occlusive member retention feature 66 is shown extending from the anchor member strut 30, in some cases the first occlusive member retention feature 66 may instead extend from the expandable frame strut 26. In some cases, the first occlusive member retention feature 66 and the second occlusive member retention feature 68 may be replicated one or more times within the space 34. In some cases, the distal anchor member 24 may include one or more first occlusive member retention features 66 extending from the anchor member strut 30 and/or one or more first occlusive member retention features 66 extending from the expandable frame strut 26.

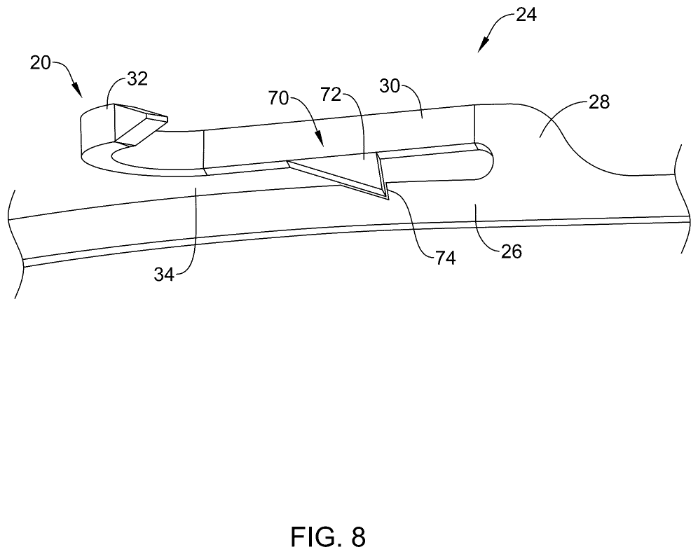

FIG. 8 shows the distal anchor member 24 as including an occlusive member retention region 70 that is adapted to help hold the occlusive member 14 relative to the expandable frame 12. The occlusive member retention region 70 may be laser cut as an integral part of the distal anchor member 24. As shown, the occlusive member retention region 70 includes a first occlusive member retention feature 72 and a second occlusive member retention feature 74. In some cases, the first occlusive member retention feature 72 includes a barb extending from the anchor member strut 30 and the second occlusive member retention feature 74 includes a complementary recess that is formed within the expandable frame strut 26. In some cases, the first occlusive member retention feature 72 extends part way into the second occlusive member retention feature 74. In some cases, the first occlusive member retention feature 72 may extend from the expandable frame strut 26 while the second occlusive member retention feature 74 may be formed within the anchor member strut 30.

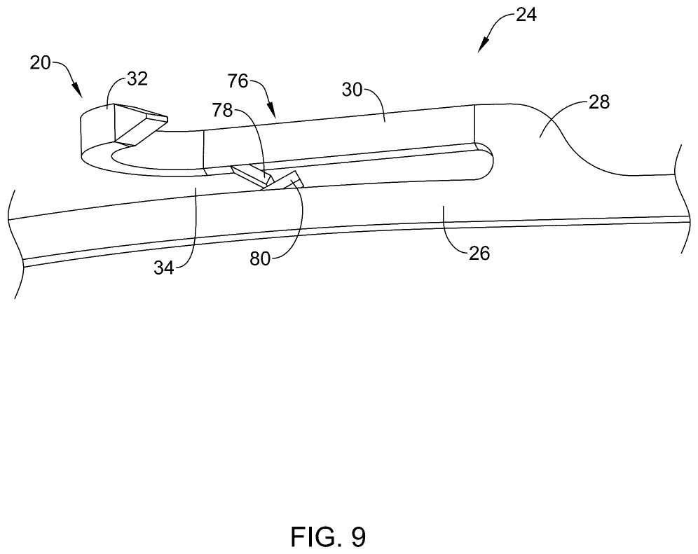

FIG. 9 shows the distal anchor member 24 as including an occlusive member retention region 76 that is adapted to help hold the occlusive member 14 relative to the expandable frame 12. The occlusive member retention region 76 may be laser cut as an integral part of the distal anchor member 24. As shown, the occlusive member retention region 76 includes a first occlusive member retention feature 78 and a second occlusive member retention feature 80. In some cases, the first occlusive member retention feature 78 has a linear profile and extends within the space 34. In some cases, the second occlusive member retention feature 80 has a linear profile and extends within the space 34. In some cases, the first occlusive member retention feature 78 and the second occlusive member retention feature 80 may be replicated one or more times within the space 34.

While FIGS. 4 through 9 provide different examples of barbs and other structures forming occlusive member retention features, it will be appreciated that these various structures may be combined in any desired fashion. These various structures may be replicated in any desired number. In some cases, one or more features shown in one of FIGS. 4 through 9 may be combined with one or more features shown in another of FIGS. 4 through 9.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device, comprising:
an expandable frame adapted to shift between a collapsed configuration and an expanded configuration, the expandable frame comprising a plurality of expandable frame struts;
an occlusive member disposed over the expandable frame; and
a plurality of anchor members, each of the plurality of anchor members coupled with a corresponding one of the plurality of expandable frame struts, each of the plurality of anchor members comprising:
an anchor member base extending laterally from the corresponding expandable frame strut;
an anchor member strut extending axially from the anchor member base and defining a space between the anchor member strut and the corresponding expandable frame strut; and
one or more pairs of opposing barbs extending into the space, each pair of opposing barbs including one barb extending into the space from the anchor member strut and one barb extending into the space from the corresponding expandable frame strut.

2. The LAAC device of claim 1, wherein for at least some of the plurality of anchor members, the anchor member strut extends at least substantially parallel with the expandable frame strut.

3. The LAAC device of claim 1, wherein for at least some of the plurality of anchor members, the anchor member struts is at least substantially coplanar with the expandable frame strut.

4. The LAAC device of claim 1, wherein at least some of the plurality of anchor members further comprise a tip portion extending from an end of the anchor member strut opposite to the anchor member base.

5. The LAAC device of claim 4, wherein for at least some of the plurality of anchor members, the tip portion curves away from the anchor member strut.

6. The LAAC device of claim 5, wherein for at least some of the plurality of anchor members, the tip portion is adapted to engage tissue when the LAAC device is implanted.

7. The LAAC device of claim 1, wherein the one or more pairs of opposing barbs comprise two pairs of opposing barbs.

8. The LAAC device of claim 1, wherein the one or more pairs of opposing barbs comprise three or more pairs of opposing barbs.

9. The LAAC device of claim 1, wherein the one or more pairs of opposing barbs are adapted to engage with the occlusive member.

10. A left atrial appendage closure (LAAC) device, comprising:
an expandable frame adapted to shift between a collapsed configuration and an expanded configuration, the expandable frame comprising a plurality of expandable frame struts;
an occlusive member disposed over the expandable frame; and
a plurality of anchor members, each of the plurality of anchor members coupled with a corresponding one of the plurality of expandable frame struts, each of the plurality of anchor members comprising:
an anchor member base extending laterally from the corresponding expandable frame strut;
an anchor member strut extending axially from the anchor member base and substantially parallel with the corresponding expandable frame strut;
a tissue engaging region extending from the anchor member strut; and
one or more pairs of opposing barbs, each pair of opposing barbs including a first barb extending towards the corresponding expandable frame strut from the anchor member strut and a second barb extending towards the anchor member strut from the corresponding expandable frame strut.

11. The LAAC device of claim 10, wherein the anchor member strut is spaced apart from the corresponding expandable frame strut such that the anchor member strut and the corresponding expandable frame strut define a space therebetween.

12. The LAAC device of claim 10, wherein the first barb and the second barb are at least substantially coplanar.

13. A left atrial appendage closure (LAAC) device, comprising:
an expandable frame adapted to shift between a collapsed configuration and an expanded configuration, the expandable frame comprising a plurality of expandable frame struts;
an occlusive member disposed over the expandable frame; and
a plurality of anchor members, each of the plurality of anchor members coupled with a corresponding one of the plurality of expandable frame struts, each of the plurality of anchor members including an occlusive member retention region including a first occlusive member retention feature and an associated second occlusive member retention feature.

14. The LAAC device of claim 13, wherein each of the plurality of anchor members comprise an anchor member strut spaced apart from the corresponding expandable frame strut.

15. The LAAC device of claim 13, wherein the first occlusive member retention feature extends at a first angle relative to one of the anchor member strut and the corresponding expandable frame strut, and the second occlusive member retention feature extends from the first occlusive member retention feature at a second angle different from the first angle.

16. The LAAC device of claim 13, wherein the first occlusive member retention feature comprises a first barb extending from the anchor member strut towards the corresponding expandable frame strut and the second occlusive member retention feature comprises a second barb extending from the corresponding expandable frame strut towards the anchor member strut.

17. The LAAC device of claim 16, wherein the first barb is axially spaced from the second barb.

18. The LAAC device of claim 13, wherein the first occlusive member retention feature comprises a barb extending from one of the anchor member strut and the corresponding frame strut, and the second occlusive member retention comprises a complementary recess formed in the other of the anchor member strut and the corresponding frame strut.

19. The LAAC device of claim 18, wherein the first occlusive member retention feature partially extends into the second occlusive member feature.

20. The LAAC device of claim 13, wherein at least some of the anchor members further comprise a tissue engaging region that curls relative to the corresponding expandable strut.
